(19) United States
(12) Patent Application Publication
(10) Pub. No.: US 2010/0069708 A1
(43) Pub. Date: Mar. 18, 2010

(54) Erectile function method and device

(76) Inventor: Kambiz Tajkarimi, Chambersburg, PA (US)

Correspondence Address: MEREK, BLACKMON & VOORHEES, LLC, 673 S. WASHINGTON ST, ALEXANDRIA, VA, 22314, US

(21) Appl. No.: 12/232,475

(22) Filed: Sep. 18, 2008

(51) Int. Cl. A61F 5/00; A61B 19/00; A61N 1/00; A61H 1/00

(52) U.S. Cl. 600/38; 128/898; 607/143; 601/46

(57) ABSTRACT

A method of improving penile erections comprises simultaneously stimulating nerves of the ventral and dorsal sides of the penis by simultaneously applying mechanical vibrations to the ventral and dorsal sides of the penis. The vibrations are applied by moving vibratory elements into contact with the penis in directions perpendicular to the ventral and dorsal sides; the vibrations are removed by moving the vibratory elements out of contact with the penis in directions perpendicular to the ventral and dorsal sides; and the vibratory elements are moved into and out of contact with the penis by the person whose penis is being stimulated. Apparatus for carrying out the method is also disclosed.

### FIELD OF THE INVENTION

The present invention relates to a method and apparatus for restoring or improving erectile and orgasmic function, especially for men whose pro-erectile cavernous nerves have been functionally impaired, such as by radical pelvic surgery (prostate, rectum, bladder), pelvic radiation (external beam or seed implant) or cryotherapy for management of prostate, rectum, or bladder cancer.

### BACKGROUND OF THE INVENTION

Prostate cancer screening programs have led to thousands of sexually healthy men being diagnosed with prostate cancer every year. Recent literature suggests that up to 80% of these cancers are confined to the prostate gland. Currently, radical prostatectomy (RP) remains the best option for management of localized prostate cancer in men with life expectancy greater than 10 years. Other options for management of localized prostate cancer include external beam radiation, radioactive seed implant, focal or total cryotherapy and active surveillance.

Erectile dysfunction (ED) after nerve-sparing prostate cancer surgery, or other nerve-sparing pelvic surgery, such as radical rectal or bladder surgery, as well as cancer related pelvic radiation or cryotherapy, is related to a certain degree with functional impairment of one or both (left, right) cavernous nerves (CN), also known as “neurovascular bundles” that travel on each side of the prostate gland to the penis. These nerves carry important pro-erectile (parasympathetic) autonomic information from the central nervous system via the sacral spinal cord to the penile erectile tissues.

Due to the anatomic proximity of cavernous nerves to the prostate, bladder, and rectum, any surgery (via cutting, thermal, stretching, etc), radiation therapy (external beam or seed implant) or cryotherapy (freezing) in this area can lead to total or partial, temporary or permanent, anatomical and functional disconnection between penile erectile tissues and pro-erection signals from the central nervous system via the cavernous nerves. This is also known as iatrogenic erectile dysfunction. The incidence of iatrogenic impotence from the above-mentioned surgical or nonsurgical interventions has been reported to be from 43% to as high as 100%. Depending on the severity of injury, this cavernous nerve paralysis (neuropraxia) can last as long as 2 years. Furthermore, the quality of returning erections is often inferior. This can have profound biological, marital, and psychological consequences in potent men undergoing surgery or other invasive intervention for cancer in this location (prostate, bladder, rectum). After such intervention, many men also suffer from urinary incontinence and orgasmic dysfunction (no orgasms, poor orgasms, painful orgasms).

Studies have shown that the dorsal nerve of the penis plays a crucial role in erections. It is especially important for rigid erections necessary for sexual intercourse. Damage to the dorsal nerve in animal studies inhibits successful sexual intercourse. The evolutionary process of the stroking movement of the male genitalia and the thrust action of sexual intercourse persistently excites the dorsal nerve fibers and perineal nerve fibers of the penis to send information to the spinal cord to provoke cavernous nerves, through complex penile and spinal connections, to release pro-erectile neurotransmitters from their nerve endings throughout corporal tissues, leading to initiation and maintenance of erection. Nitric oxide released by the nerve endings of the cavernous nerves (NNOS) has been proposed to be important in the initiation of erection, the initiation being known to be of short duration. However, the action of filling of penile blood vessels (shear force) initiated by cavernous nerves leads to subsequent release of nitric oxide by endothelial cells that line penile blood vessels. Endothelial-driven nitric oxide synthase (eNOS) activity is responsible for a release of nitric oxide that aids in maintaining erections after the initiation.

Dorsal nerve excitation also stirs up pudendal nerve activity through the bulbo-cavernosus reflex to contract the deep muscles of the penis to make penis extra-rigid adequate for sexual intercourse. Several studies suggest that stimulation of the perineal nerve initiates penile erections by means of cavernous muscle contractions (skeletal muscles of the penis) subsequent to corporal pressure elevation. Additionally, dorsal and perineal nerve stimulation sends sensory information to erotic centers of the cerebral cortex, thalamus, auditory, visual, olfactory and memory centers to potentiate a very complex cascade of biochemical and neurophysiological events that lead to sexual excitation and penile erection.

The pudendal nerve and its branches take a different anatomical path from cavernous nerves on their way to and from the penis, anal sphincter, and urethral sphincter. This path is safely away from the surgical field during pelvic surgery and other interventions. Therefore, the pudendal nerve and its branches (DNP and PN) remain intact and continue penile communication with the brain, spinal cord and the pelvic plexus of nerves. In fact, if both cavernous nerves are injured or disconnected, the pudendal nerve system becomes the only intact neuronal pathway between penile tissues and the brain and spinal cord. Furthermore, scientists have discovered multiple connections between branches of the dorsal nerve of the penis (DNP) and the cavernous nerve (CN), at regular intervals, within the sympathetic chain, spinal cord and throughout the tissues of the penile root and shaft. Although much of this connectivity remains a mystery, pro-erectile nitric oxide synthase (NOS) activity has been discovered in DNP-CN junctions. In the rat model, NOS-containing nerves are able to regenerate after cavernous nerve disconnection.

The penile erectile tissue, specifically the corpus cavernosum smooth musculature and the smooth muscles of the arteriolar and arterial walls, plays a key role in the erectile process. A normal flaccid penis is in a moderate state of tonic contraction, allowing only a small amount of arterial blood for nutritional purposes. Normal involuntary erections mediated by the central nervous system during sleep and erotic-mediated sexual stimulus result in release of neurotransmitters (acetyl-choline, nitric oxide and others) from cavernous nerve terminals and cells lining blood vessels (endothelial cells) that result in a complex cascade of anatomic and biochemical events that fill penile erectile tissues with oxygenated arterial blood, expanding penile sinusoidal tissues, and passive compression of outgoing venous flow which ultimately results in erections.

Such cavernous nerve-mediated initiation of pro-erectile nerve stimulus is vital for daily replenishment of corporal (penis) oxygen supply and other metabolic needs in healthy men. Some scientists believe that disruption of such frequent cavernous nerve mediated oxygen and nutrition delivery, sometimes multiple times during the night, can have numerous and lasting detrimental consequences. Several histological analyses have demonstrated multiple chronic hypoxia (poor oxygen state) related effects: decreased smooth muscle number, cell-cell contact, venous leakage, collagen deposition, fibrosis, decreased penile weight and length, and additional irreversible and harmful effects to penile tissues after injury to the cavernous nerves in humans and animals.

Cavernous nerve paralysis (neuropraxia) caused by above-mentioned surgical and nonsurgical interventions can last as long as 2 years, even more. Slowly, natural nerve regeneration systems of the body attempt to reestablish this connection after injury. However, even with some gradual restoration of cavernous nerve functional connectivity, there may be little or no healthy and responsive penile tissues to permit adequate fullness firm enough for copulation. Recently, urologists have proposed a proactive approach to use of oral medications (i.e. phosphodiesterase inhibitors, etc.) or an intraurethral insert or lintercavernosal injection treatments (alprostadil), or neuromodulation, or vacuum-assisted devices, while post-intervention neuropraxia recovery is awaited. Unfortunately, the prohibitive costs of medications, poor response, and pain from injectables often lead to high dropout rates.

In the human glans penis are millions of sensory nerve terminations: free nerve endings and corpuscular receptors. The free nerve endings are derived from thin myelinated Aδ and unmyelinated C fibers. The nerve fibers from the receptors converge to form bundles of the dorsal nerve of the penis, which joins other nerves, including the perineal nerve to become the pudendal nerve. The latter enters the spinal cord via the S2-S4 roots to terminate on spinal neurons and interneurons in the central gray region of the lumbosacral segment. Activation of these sensory neurons sends messages of pain, temperature, and touch by means of spinothalamic and spinoreticular pathways to the thalamus and sensory cortex for sensory perception. They are also the basis for the bulbocavemosus reflex. Onuf's nucleus in the second to fourth sacral spinal segments is the center of somatomotor penile innervation. Outgoing nerves from Onuf's nucleus travel as pudendal nerve to innervate the ischiocavemosus and bulbocavemosus muscles of the penis. Contraction of the ischiocavemosus muscle produces the rigid-erection phase. Rhythmic contraction of the bulbocavemosus muscle is necessary for ejaculation. Persistent stimulation of the pudendal nerve and its branches can maintain and strengthen the striated muscles of ischio- and bulbospongiosus muscles, which, in addition, can enhance the rigidity of erections. A relationship exists between the dorsal nerve of the penis afferents and motoneurons destined to pelvic striated muscles. Stimulation of the DNP elicits firing on the motor PN in rats.

As mentioned earlier herein, the afferent (from penis to spinal cord) DNP and the efferent (from spinal cord to penis) motor pudendal nerve are the basis of the bulbocavemosus reflex, and the bulbocavemosus reflex is necessary for copulation to succeed. Studies have shown that local anesthesia of DNP in rats inhibits the act of copulation, as the rats are unable to insert the penis into the vaginal orifice. Studies in man and in animals have shown that contraction of ischiocavemosus muscle by stimulation of the perineal nerve creates mechanical compression of the proximal part of erectile tissue. This super-rigidity occurs as a reflex response to stimulation of the glans during coitus. In post prostatectomy patients, this reflex is under-utilized, since the penis is often not rigid enough to penetrate the female to initiate the reflex.

Persistent vibratory stimulation can activate this reflex, and there is anecdotal evidence of sexually healthy men who have performed the method according to the present invention prior to sexual intercourse and report unexpectedly rigid erections that last longer than usual.

As was described before, the spinal cord is the center of distribution of efferent autonomic (parasympathetic, sympathetic) and somatic pathways to the penis and related striated muscles (urethral and anal sphincter). It receives peripheral information from the glans penis and corpora from the dorsal nerve of the penis (DNP), the cavernous nerve, and the perineal nerve (PN). In animals, stimulation of penile afferents activates spinal neurons in the dorsal horn, dorsal gray commissure in the lumbosacral region. The dorsal gray commissure represents an integration site for both visceral and somatic afferents. Electrical stimulation of dorsal nerve of penis (DNP) in the rat elicits firing of pelvic, hypogastric, and cavernous nerves. Similarly, natural aging and common medical disease processes (chronic hypertension, diabetes mellitus, peripheral vascular disease and atherosclerosis) negatively impact penile erections by, for example, impaired vascular flow, decreased elasticity of erectile tissues, smooth muscle breakdown, venous leakage and poor nerve function (neuropathy).

### SUMMARY OF THE INVENTION

By the present invention, nerve endings of the dorsal nerve of the penis (DNP) and the perineal nerve (PN), which are branches of the pudendal nerve, are stimulated by using simultaneous vibratory stimulation of both the dorsal and ventral sides of the penile shaft and the glans penis, as well as the frenulum. The skin of the penile shaft, the frenulum (lower tip of penis) and the glans penis are innervated by sensory fibers of the dorsal nerve of the penis and the perineal nerve, which carry separate and distinct sensory and autonomic fibers to and from the spinal cord and brain.

In addition, the perineal branch of the pudendal nerve carries somatic (motor) stimulation from the spinal cord to the skeletal muscles of the penis (bulbous spongiosum, and ischiocavernosum), external urethral sphincter, and anal sphincter. The method according to the present invention stimulates fibers of the perineal nerve in order to strengthen the muscles of the external urethral sphincter and external anal sphincter and the skeletal muscles of the penis. This stimulation improves urinary and fecal continence over time.

Studies have shown that there exist dual sensory innervations of the penis. Several published genital electrophysiologic tests of human and animal subjects have revealed evoked cortical responses after perineal nerve stimulation independent of dorsal nerve of the penis.

Apparatus according to the present invention is specifically designed to simultaneously stimulate both sensory systems of the dorsal nerve of penis (DNP) and the perineal nerve (PN).

The present invention targets the DNP and the DNP-CN junctions to communicate with damaged cavernous nerves distal to the area of injury in an effort to stimulate retrograde regeneration and up-regulation of nitric oxide synthase activity at the DNP-CN junction.

The present invention targets the sensory nerves of the penis to stimulate filling of blood vessels by a vibratory mechanism. This vibratory mechanism increases shear force within the blood vessels, leading to endothelial-derived nitric oxide release that can lead to erections independent of cavernous nerves.

Dorsal and perineal nerve stimulation helps activity in alternative pathways of erections that are thought to be independent of cavernous nerve. Persistent stimulation of the central nervous pro-erectile pathways by the method of the present invention may also aid in enhancing recovery and regeneration of injured cavernous nerves via anterograde regeneration.

The present invention is a neuromodulatory method and apparatus that takes advantage of the body's natural neuronal connections and other neurophysiological processes at multiple steps and levels. It excites the sensory nerves of the skin and deeper tissues of the penis by a vibratory method. The excitation can be maximized by using two separate motors each with a capacity for generating vibrations of adjustable frequency and amplitude.

The present invention can be used shortly after surgical or invasive intervention as a form of safe and non-invasive erectile rehabilitation regimen of exercises for several minutes (for example, 5-10 min.) at regular intervals in the range of twice a day to twice a week, which can be performed for weeks to months, or indefinitely, depending on the severity of injury and the response to treatment. Persistent specific anatomic vibratory stimulation of intact sensory nerves of the penis increases the activity of local, spinal, and cerebral pathways. Simultaneous stimulation of dorsal and perineal nerve branches at regular intervals can up-regulate activity at cavernous nerve-dorsal nerve junctions, produce daily penile fullness that can help deliver arterial blood and oxygen and other essential metabolites, help preserve erectile tissue integrity and penile length, encourage natural release of anti-inflammatory and neuromodulatory chemicals, and amplify somatic/autonomic activity that may strengthen external urethral and anal sphincter muscle tone and produce orgasms. The present invention takes advantage of the body's natural systems to preserve erectile tissue integrity during cavernous nerve neuropraxia, prevent erectile tissue destruction, and enhance recovery of erectile function by accelerating cavernous nerve regeneration by natural neuromodulation.

The method of the present invention can be used in men whose cavernous nerves have been completely and permanently destroyed to preserve their erectile tissue integrity and length in anticipation of further intervention, such as penile prosthetic implants. The method of the present invention can also be used for obtaining sexual stimulation, orgasm and ejaculation. The method of the present invention can further be used for retarding age-related erectile dysfunction and degeneration and age-related disuse atrophy and negative changes in penile nervous and erectile tissue. The method of the present invention can also be used for retarding chronic disease-related erectile dysfunction and degeneration and negative changes in penile nervous and erectile tissue. The chronic diseases include diabetes mellitus, chronic hypertension, peripheral vascular disease, high cholesterol, and atherosclerosis.

There is general consensus in the medical and psychological literature that repeated genital vibratory stimulation is safe. Millions of women use frequent genital/clitoral vibratory stimulation for purposes of relaxation and pleasure. In connection with the present invention, the safety and satisfaction of daily vibratory stimulation using a conventional massager has been assessed in 20 men after prostate surgery for up to a year. There have been no reports of side effects or complications. Patients report high satisfaction and penile fullness and/or erections, and orgasms after each use.

Penile vibration of men with spinal cord injury for ejaculatory dysfunction has exposed many profound and unanticipated additional benefits: relief of lower extremity spastic muscle contractions, increased bladder capacity, decreased bladder hyperactivity, increased continence and reflexogenic erections. The apparatus and method according to the present invention can help all aging men with poor or worsening penile tissue health, diabetics, vascular disease due to chronic hypertension, peripheral vascular disease and high cholesterol, and atherosclerosis. The method and apparatus according to the present invention can be used in combination with other erectile aid modalities available today or in the future. The method and apparatus according to the present invention is safe, with no systemic side-effects.

Furthermore, the present invention can be used on-demand (prior to sexual activity) to enhance, that is, potentiate, tissue response to oral, intraurethral or injectable pro-erection medication, including phosphodiesterase inhibitors, prostaglandin E-1 (alprostadil) and others. Such medications work only when the person taking them is sexually stimulated, such as with foreplay prior to sexual intercourse. Such medications aid in dilating arterial vessels by varying mechanisms. The method according to the present invention maximally stimulates sensory nerves of the penis that, in return, augment tissue response to such medication. The method according to the present invention may allow men to use lower doses of such medication to be taken, with higher erectile tissue response resulting, and may prevent patients from seeking higher doses of drugs or other more invasive therapies (injectables or invasive penile prosthetic surgery). The present invention may help patients to have satisfactory erections adequate for penetration with a reduced amount of medications and, as a result, decrease systemic side effects from such drugs.

The stimulation can be self-performed conveniently with one hand in a standing, seated, or supine position, or can be carried out by another person.

The on-demand (prior to or during sexual activity) application of the present invention is not limited to persons taking pro-erectile medication and includes simultaneous vibratory stimulation of both the dorsal and ventral sides of the penis for sexual stimulation, orgasm, and/or ejaculation during sexual activity, masturbation or foreplay.

Persistent vibratory stimulation can activate the reflex response to stimulation of the glans during coitus, and there is anecdotal evidence of sexually healthy men who have performed the method according to the present invention prior to sexual intercourse and report unexpectedly rigid erections that last longer than usual. The same autonomic response could be elicited by stimulation of cutaneous as well as visceral afferents.

This suggests that stimulation of DNP by the method according to the present invention can clearly communicate with, and perhaps increase activity within, the cavernous nerve. Such communication, with the possible increased activity, is one basis of the penile rehabilitation method of the present invention to enhance recovery of erectile function by augmenting activity within an injured cavernous nerve and aid in natural regeneration and sprouting of new nerves to repair pelvic nerve circuitry after pelvic surgery or other invasive intervention.

The apparatus and method according to the present invention also targets patients affected by natural aging and common medical disease processes for vibratory neuromodulation to treat and retard deleterious effects of aging and such chronic medical diseases on penile erectile function.

### BRIEF DESCRIPTION OF FIGURES AND DRAWINGS

FIG. 1 is a schematic vertical cross section showing the anatomy of pelvic nerve circuitry in relation to the penis;

FIG. 2 is a schematic cross section of the anatomy of the penis;

FIG. 3a is a schematic illustration of the erectile tissue of a flaccid penis;

FIG. 3b is a schematic illustration of the erectile tissue of an erect penis;

FIG. 4a is a schematic perspective from the left side and top of a penis showing the nerve distribution of the dorsal nerve;

FIG. 4b is a schematic perspective from the left side and bottom of a penis showing the nerve distribution of the perineal nerve;

FIG. 5 is a perspective view from the front, top and left side of an apparatus according to the present invention;

FIG. 6 is a perspective of the device of FIG. 5, with portions of a casing removed;

FIG. 7 is an enlarged perspective of the hinge area of the device of FIG. 6; and

FIG. 8 is a perspective view of the apparatus according to the present invention in position to engage a penis (shown schematically).

### DETAILED DESCRIPTION OF THE PREFERRED EMBODIMENTS

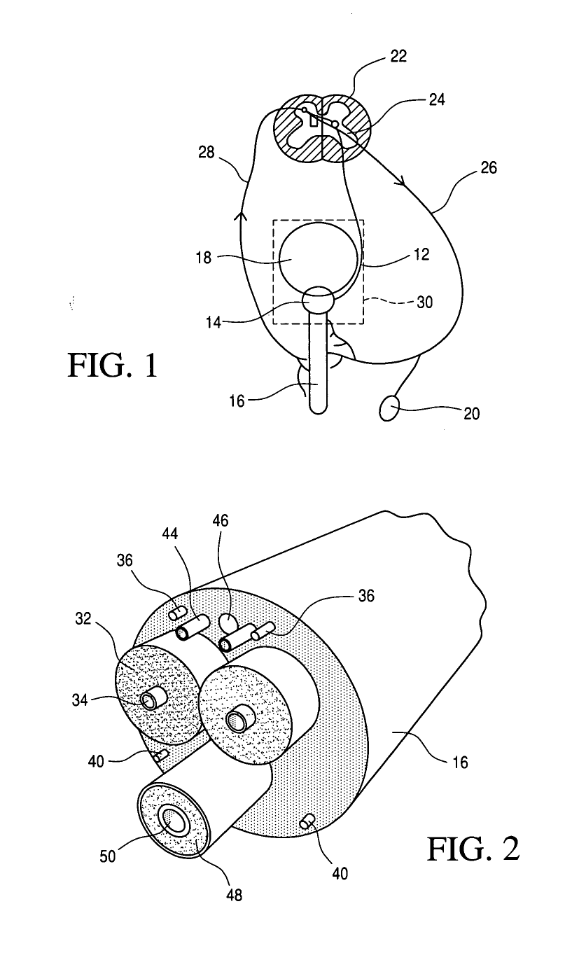

As can be appreciated from FIG. 1, a cavernous nerve (CN) 12, also known as a “neurovascular bundle”, extends on each side of the prostate gland 14 to the penis 16. Erectile dysfunction (ED) after nerve-sparing prostate cancer surgery, or other nerve-sparing pelvic surgery, such as radical rectal or bladder surgery, as well as cancer related pelvic radiation or cryotherapy, is related to a certain degree with functional impairment of one or both (left, right) of the cavernous nerves 12. These nerves carry important proerectile (parasympathetic) autonomic information from the central nervous system via the sacral spinal cord to the penile erectile tissues. The bladder 18, the anal sphincter 20, the sacral spinal cord 22, Onuf's nucleus 24, the pudendal nerve motor branches 26, and the pudendal nerve sensory branches 28 are also shown schematically in FIG. 1, as is a box 30 representing the area of potential iatrogenic injury to the cavernous nerves 12. Surgery, radiation therapy or cryotherapy in the area of the cavernous nerves 12, generally within the box 30 in FIG. 1 can lead to total or partial, temporary or permanent anatomical and functional disconnection between penile erectile tissues and pro-erection signals from the central nervous system via the cavernous nerves.

As can also be appreciated from FIG. 1, the branches 26 and 28 of the pudendal nerve take a different anatomical path on their way to and from the penis 16, anal sphincter 20, and urethral sphincter, a path that is safely away from the surgical field during pelvic surgery and other interventions. The pudendal nerve branches 26 and 28 enter the spinal cord 22 via the S2-S4 roots to terminate on spinal neurons and interneurons in the central region of the lumbosacral segment. As a result of their location, the pudendal nerve motor branches 26, and the pudendal nerve sensory branches 28, including the terminal branches of the pudendal nerve sensory branches 28, namely, the dorsal nerve of the penis (DNP) and the perineal nerve (PN), remain intact after pelvic surgery and other interventions and continue penile communication with the brain, spinal cord and the pelvic plexus of nerves.

As can be seen from the schematic cross section of FIG. 2, the penile erectile tissue, specifically the corpus cavemosum smooth musculature 32 and the smooth muscles of the walls of the arteries 34 and of their small terminal branches, plays a key role in the erectile process. Also shown in FIG. 2 are the left and right branches, respectively, of the dorsal nerve 36, the left and right branches, respectively, of the perineal nerve 40, the dorsal arteries 44, the dorsal vein 46, the corpus spongiosum 48 and the urethra 50.

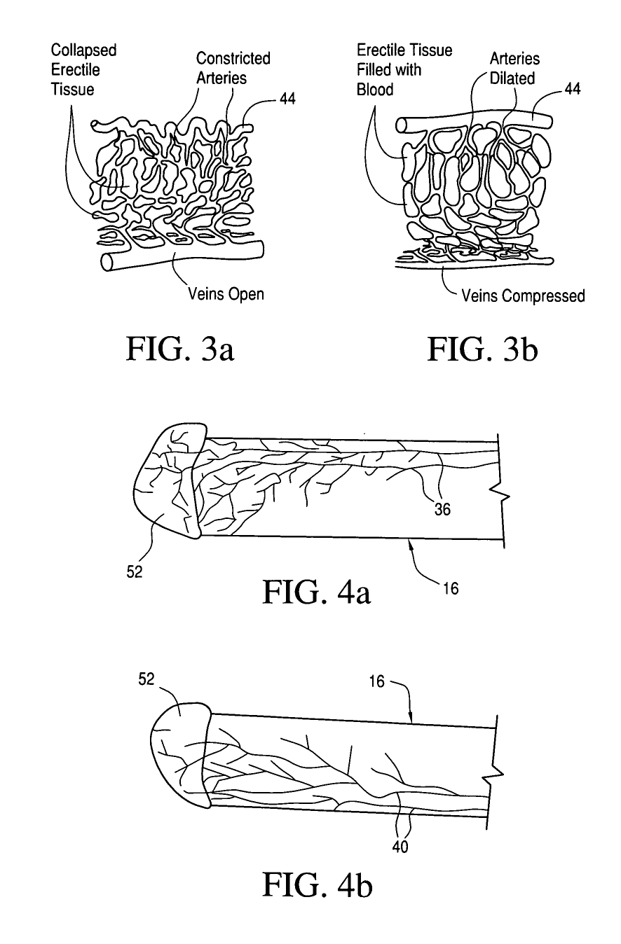

A normal flaccid penis is in a moderate state of tonic contraction, allowing only a small amount of arterial blood for minimal nutritional purposes. FIG. 3a shows a portion of the erectile tissue, arteries 44 and veins of a normal flaccid penis, in which the erectile tissue is collapsed, the arteries are constricted, and the veins are open. FIG. 3b shows a portion of the erectile tissue, arteries and veins of a normal erect penis, in which the erectile tissue is filled with blood, the arteries are dilated, and the veins are compressed.

By the present invention, nerve endings of the dorsal nerve 36 of the penis 16 (DNP) and the perineal nerve 40 (PN), which are branches of the pudendal nerve and are shown in FIGS. 4a and 4b, respectively, are stimulated by using simultaneous vibratory stimulation of both the dorsal and ventral sides of the penile shaft, preferably including the glans penis 52 (lower tip of penis), as well as the frenulum. The skin of the penile shaft, the frenulum and the glans penis are innervated by sensory fibers of the dorsal nerve 36 of the penis and the perineal nerve 40, which carry separate and distinct sensory and autonomic fibers to and from the spinal cord and brain.

The apparatus according to the present invention is specifically designed to simultaneously stimulate both sensory systems of the dorsal nerve of penis (DNP) and the perineal nerve (PN).

A method of obtaining or improving penile erections according to the present invention includes simultaneously stimulating the nerves of the ventral and dorsal sides of the penis, that is, the perineal nerve 40 and the dorsal nerve 36, by simultaneously applying mechanical vibrations to the ventral and dorsal sides of the penis. The vibrations are applied by moving vibratory elements into contact with the ventral and dorsal sides of the penis in directions perpendicular to the ventral and dorsal sides of the penis. The vibrations are removed by moving the vibratory elements out of contact with the ventral and dorsal sides of the penis in directions perpendicular to the ventral and dorsal sides of the penis. The vibratory elements are moved into and out of contact with the penis by the person whose penis is being stimulated.

The vibrations are applied by moving the vibratory elements toward one another in directions perpendicular to the ventral and dorsal sides of the penis and, before the vibratory elements are moved toward one another and into contact with the penis, the vibratory elements are moved laterally with respect to the penis from positions in which the penis does not lie between the vibratory elements to positions in which the penis lies between the vibratory elements.

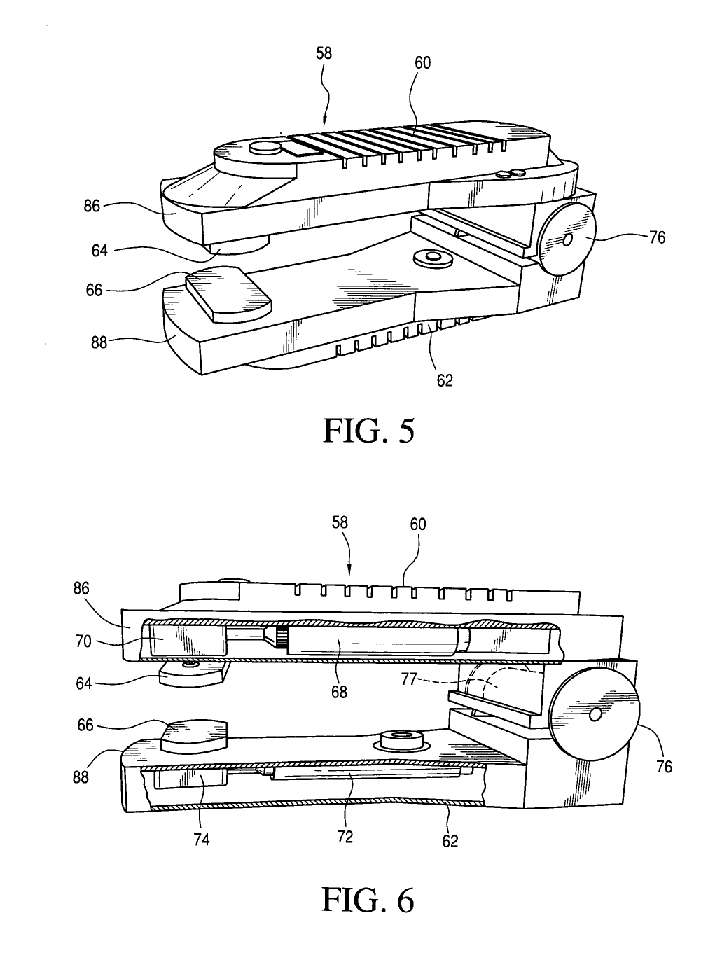

As can be seen from FIGS. 5-7, a device 58 according to the present invention for performing the method includes a first member 60, a second member 62, a first vibratory element 64 positioned on the first member, and a second vibratory element 66 positioned on the second member. An arrangement is provided for vibrating the first and second vibratory elements 64 and 66. In the illustrated embodiment, as can best be seen from FIG. 6, the arrangement for vibrating includes an electric motor 68 and a vibration-creating mechanism 70 for vibrating the first vibratory element 64, as well as an electric motor 72 and a vibration-creating mechanism 74 for vibrating the second vibratory element. In the illustrated embodiment, the first and second vibratory elements 64, 66 vibrate, but the first and second members 60, 62 do not vibrate.

U.S. Pat. No. 6,027,444 to Franck, which is hereby incorporated by reference in its entirety, discloses a vibratory element, motor and vibration-creating mechanism that are suitable for use in the device according to the present invention, the present invention including two vibratory elements, motors and vibration-creating mechanisms. U.S. Pat. No. 6,027,444 also discloses a frequency regulating device and an amplitude regulating device for regulating the frequency and the amplitude, respectively, of the upward and downward movement of the vibratory element. One of the amplitude regulating devices is suitable for each of the vibratory elements of the device according to the present invention, and one of the frequency regulating devices is suitable for both vibratory elements of the present invention.

A connection between the first and second members 60, 62 enables the first and second members to be moved toward and away from one another, and a mechanism, for example, a spring, resiliently biases the first and second members away from one another with a force that is able to be overcome with one hand. In the illustrated embodiment the connection is shown schematically as a hinge connection 76, and a battery 77 is provided in the area of the hinge connection to power the motors 68 and 72.

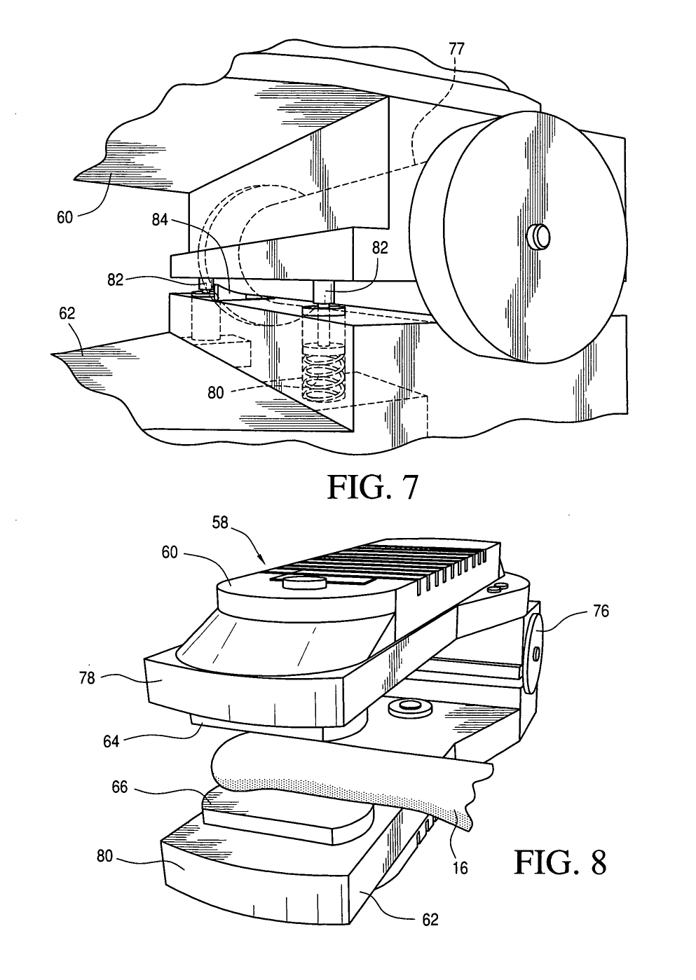

As can be seen from FIG. 7, springs 80 (one of which is shown) positioned in the second member 62 resiliently bias contact members 82 toward the first member 60 to contact a portion of the first member. The vibratory elements 64, 66 are actuated in response to the vibratory elements reaching predetermined positions relative to the penis in the following way. As the first member 60 and the second member 62 are made to approach one another against the force of the springs 80, such as by the force of a hand, a portion of the first member engages an actuator 84 of a switch (not shown), the actuator being mounted on and projecting up from the second member. The switch is in an electric circuit with the battery 77 and the electric motors 68 and 72. As a result of a portion of the first member 60 engaging the actuator 84, the electric circuit is closed and the motors 68 and 72 are turned on. Release of the force of the hand allows the springs 80 to move the first and second members 60, 62 apart and thereby shut off the motors 68, 72. The actuation of the switch is calibrated to occur at about the point that both vibratory elements 64, 66 of the device contact a flaccid penis.

The first vibratory element 64 is positioned to contact the dorsal side of the penis when the second vibratory element 66 contacts the ventral side of the penis. The hinge connection 76 limits the movement of the first and second members 60, 62 away from one another to predetermined relative positions in a conventional way. Each of the first and second members 60, 62 has a distal side facing away from the other of the first and second members, and the distance between the distal sides when the first and second members are in the predetermined positions is less than the distance between the thumb and fingers of a man's open hand. As a result, a man is able to hold the device with one hand positioned to the side of the penis, move the device laterally with respect to his penis with that hand, and move the first and second members 60, 62 toward one another, against the force of the springs 80, with the same hand.

In the illustrated embodiment, the first vibratory element 64 is adapted to be in contact with a penis. More specifically, in the illustrated embodiment, the first vibratory element 64 protrudes from the first member 60 in a direction toward the second vibratory element 66 and toward the penis, as can be seen from FIG. 8. Similarly, the second vibratory element 66 protrudes from the second member 62 in a direction toward the first vibratory element 64 and toward the penis. The first vibratory element 64 is adapted to be in contact with the penis simultaneously with the second vibratory element 66.

Each of the first member 60 and the second member 62 has a distal end, 86 and 88 respectively, spaced from the hinge connection 76. In view of this structure, the device 58 can be moved laterally with respect to the penis (perpendicular to the length of the penis) to receive the penis between the first vibratory element 64 and the second vibratory element 66 and between the hinge connection 76 and the distal ends 86, 88 of the first and second members.

The vibratory elements 64, 66 vibrate in directions perpendicular to the ventral and dorsal sides of the penis. For maximum effect, the lower of the vibratory elements, the first vibratory element 66, is moved into contact with the frenulum of the penis.

In one exercise employing the method according to the present invention, the penis can be held extended with one hand (left or right), and the device 58 held and moved with the other hand such that the upper and lower vibratory elements 64, 66 gently touch the skin of the penis from above and below. During the exercise, only the vibratory elements 64, 66 contact and stimulate the skin of the penis. The device 58 can be held in one place during the entire exercise or moved to different areas of skin of the penis to minimize or maximize sensory response as desired. It can be moved along the penis in a sliding fashion to increase or decrease areas of sensation. The vibrations of the vibratory elements 64, 66 can be stopped and restarted, and their frequency and amplitude adjusted as desired without removing the device 58 from the penis. When it is desired to remove the device 58 from the penis, the device can be removed quickly and easily by releasing the force of the hand and moving the device laterally with respect to the penis by using the same hand. If desired, the device 58 can be removed by, alternatively or in combination with lateral movement, moving the device longitudinally with respect to the penis by using the hand holding the device.

The device 58 can be held in place or moved as needed for maximal response. In an exercise using the method according to the present invention, vibrations are applied to the penis for a period of several minutes, preferably in the range of approximately 3 minutes to approximately 10 minutes, or until ejaculation, whichever occurs first. A period of less than 3 minutes may be sufficient if ejaculation occurs, or erection is achieved, during that shorter time.

The exercises are performed at regular intervals, preferably in the range of twice a day to twice a week. The frequency and duration of exercises depend on the extent of injury to the cavernous nerve, age, pre-surgery sexual function and individual response. The exercises are continued until subjective and objective goals are achieved.

As an alternative to the use of the method according to the present invention as a series of exercises, the method can be performed “on demand” prior to sexual activity, either by itself or in combination with other methods of achieving erections, such as oral, intraurethral, or intracavernosal medications, for example, phosphodiesterase inhibitors, prostaglandin E-1, and others. Phosphodiesterase inhibitors currently on the market include sildenafil, tadalafil and vardenafil, as well as udenafil and avanafil. Intraurethral or intracavemosal injections include prostaglandin E-1 (alprostadil). It is expected that the method of the present invention can be used with other phosphodiesterase inhibitors, intraurethral or intracavernosal injections and other medications that may become available in the future.

When the method is performed “on demand” prior to sexual activity in combination with pro-erection medications, a smaller dose of the pro-erection medication can be administered than would otherwise be administered. For example, a smaller dose of the pro-erection medication can be administered than is recommended for erectile response by the person's physician or the manufacturer of the medication.

The method of the present invention is especially beneficial in men who have undergone prostate cancer surgery, or other radical surgery in the pelvis (bladder, rectum), or pelvic radiation therapy for cancer (external beam or radioactive seed implant) or prostate cryotherapy. The method of the present invention enhances recovery and improves erectile function, continence, and orgasmic function in this population. The method is beneficial for men suffering from erectile dysfunction from various causes, including psychogenic, neurogenic, and organic causes.

When used with pro-erectile medications, the method of the present invention is utilized from about 10 minutes up to about 36 hours after administration of the medication, depending on the onset of action, duration, and the half-life of the particular medication. The method of the present invention can also be performed before or during sexual activity as a form of foreplay or self-stimulation.

It will further be appreciated by those skilled in the art and it is contemplated that variations to the embodiments illustrated and described herein may be made without departing from the spirit and scope of the present invention. Accordingly, it is intended that the foregoing description is illustrative only, and the true spirit and scope of the invention will be determined by the appended claims.

1. A method of improving penile erections, comprising:
simultaneously stimulating nerves of the ventral and dorsal sides of the penis by simultaneously applying mechanical vibrations to the ventral and dorsal sides of the penis,
wherein the vibrations are applied by moving vibratory elements into contact with the ventral and dorsal sides of the penis in directions perpendicular to the ventral and dorsal sides of the penis,
wherein the vibrations are removed by moving the vibratory elements out of contact with the ventral and dorsal sides of the penis in directions perpendicular to the ventral and dorsal sides of the penis, and
wherein the vibratory elements are moved into and out of contact with the penis by the person whose penis is being stimulated.

2. The method of claim 1, wherein the vibratory elements vibrate in directions perpendicular to the ventral and dorsal sides of the penis.

3. The method of claim 1, wherein one of the vibratory elements is moved into contact with the frenulum.

4. The method of claim 1, wherein the method is performed at regular intervals.

5. The method of claim 1, wherein the method is performed at regular intervals in the range of twice a day to twice a week.

6. The method of claim 1, further comprising administering pro-erection medication.

7. The method of claim 6, wherein a smaller dose of the pro-erection medication is administered than is recommended by the manufacturer of the medication for use in the absence of simultaneously applying mechanical vibrations to the ventral and dorsal sides of the penis.

8. The method of claim 7, wherein the pro-erection medication is a phosphodiesterase inhibitor.

9. The method of claim 6, wherein the vibrations are applied from about 10 minutes to about 36 hours after the pro-erection medication is administered.

10. The method of claim 1, wherein the vibrations are applied by moving the vibratory elements toward one another in directions perpendicular to the ventral and dorsal sides of the penis, and wherein, before the vibratory elements are moved toward one another and into contact with the penis, the vibratory elements are moved laterally with respect to the penis from positions in which the penis does not lie between the vibratory elements to positions in which the penis lies between the vibratory elements.

11. The method of claim 10, wherein the vibratory elements are secured to members that are connected to one another such that the members are able to be moved toward one another with one hand.

12. The method of claim 11, wherein the members are hinged to one another.

13. The method of claim 11, wherein the members are held by a hand positioned at the side of the penis.

14. The method of claim 11, wherein the members are resiliently biased away from one another by a force that is able to be overcome with one hand.

15. The method of claim 10, wherein the vibratory elements are actuated in response to the vibratory elements reaching predetermined positions relative to the penis.

16. The method of claim 15, wherein the vibratory elements reach the predetermined positions due to force applied by a hand, and the vibratory elements are inactivated upon release of the force applied by the hand.

17. The method of claim 1, wherein the vibrations are stopped and restarted without removing the vibratory elements from the penis.

18. The method of claim 1, wherein at least one of the frequency and amplitude of the vibrations is adjusted without removing the vibratory elements from the penis.

19. The method of claim 1, wherein the vibratory elements are held in one place each during the applying of the vibrations.

20. The method of claim 1, wherein the vibratory elements are moved from one place each during the step of applying of the vibrations to at least one other place each in order to increase response.

21. The method of claim 1, wherein the vibrations are applied for a period of approximately 3 to approximately 10 minutes or until ejaculation, whichever occurs first.

22. The method of claim 1, wherein the method is performed immediately prior to sexual activity.

23. The method of claim 1, wherein the method is performed in combination with another method of achieving an erection.

24. The method of claim 1, wherein the method is performed on a man whose cavernous nerves have been completely and permanently destroyed, in order to preserve the integrity and length of erectile tissue of the man.

25. A method of improving penile erection function in patients having functional impairment of cavernous nerves, comprising:
simultaneously stimulating the nerves of the ventral and dorsal sides of the penis by simultaneously applying mechanical vibrations to the ventral and dorsal sides of the penis,
wherein the vibrations are applied by moving vibratory elements in directions perpendicular to the ventral and dorsal sides of the penis into contact with the ventral and dorsal sides of the penis,
wherein the vibrations are removed by moving the vibratory elements in directions perpendicular to the ventral and dorsal sides of the penis out of contact with the ventral and dorsal sides of the penis, and
wherein the vibratory elements are moved into and out of contact with the penis by the person whose penis is being stimulated.

26. A method of retarding age-related erectile dysfunction and degeneration and age-related disuse atrophy and negative changes in penile nervous and erectile tissue, comprising:
simultaneously stimulating the nerves of the ventral and dorsal sides of the penis by simultaneously applying mechanical vibrations to the ventral and dorsal sides of the penis,
wherein the vibrations are applied by moving vibratory elements in directions perpendicular to the ventral and dorsal sides of the penis into contact with the ventral and dorsal sides of the penis,
wherein the vibrations are removed by moving the vibratory elements in directions perpendicular to the ventral and dorsal sides of the penis out of contact with the ventral and dorsal sides of the penis, and
wherein the vibratory elements are moved into and out of contact with the penis by the person whose penis is being stimulated.

27. A method of improving penile erections, comprising:
simultaneously stimulating the nerves of the ventral and dorsal sides of the penis by simultaneously applying vibrations to the ventral and dorsal sides of the penis; and
administering pro-erection medication to the person to whose penis the vibrations are being applied.

28. Apparatus for improving penile erections, comprising:
a first member;
a second member;
a first vibratory element positioned on the first member;
a second vibratory element positioned on the second member;
means for vibrating the first and second vibratory elements;
a connection between the first and second members enabling the first and second members to be moved toward and away from one another; and
a mechanism resiliently biasing the first and second members away from one another,
wherein at least one of the first member and the first vibratory element is adapted to contact a penis simultaneously with at least one of the second member and the second vibratory element.

29. The apparatus of claim 28, wherein each of the first member and the second member has an end spaced from the connection,
whereby the apparatus can be moved laterally with respect to the penis to receive the penis between the first member and the second member and between the connection and said end of each of the first member and the second member.

30. The apparatus of claim 28, further comprising a first vibratory element and a second vibratory element, and an arrangement for actuating the means for vibrating when the first vibratory element and the second vibratory element contact a penis.

31. The apparatus of claim 28, wherein at least one of the first member and the first vibratory element is positioned to contact the dorsal side of the penis when at least one of the second member and the second vibratory element contacts the ventral side of the penis.

32. The apparatus of claim 29, wherein the connection limits the movement of the first and second members away from one another to predetermined relative positions, and wherein each of the first and second members has a distal side facing away from the other of the first and second members, and the distance between the distal sides when the first and second members are in said predetermined positions is less than the distance between the thumb and fingers of a man's open hand, whereby a man is able to hold the apparatus with one hand and move the first and second members toward one another with the same hand.

33. The apparatus of claim 30, wherein the first vibratory element is adapted to contact a penis simultaneously with the second vibratory element, and wherein the first and second members do not vibrate.

34. The apparatus of claim 28, further comprising a switch actuator positioned to be actuated when the first and second members are moved toward one another by a predetermined distance, wherein the vibratory elements are actuated in response to a switch actuator being actuated as the upper and lower members are moved toward one another.

35. A method of obtaining sexual stimulation, orgasm, and ejaculation comprising:
simultaneously stimulating the nerves of the ventral and dorsal sides of the penis by simultaneously applying mechanical vibrations to the ventral and dorsal sides of the penis,
wherein the vibrations are applied by moving vibratory elements in directions perpendicular to the ventral and dorsal sides of the penis into contact with the ventral and dorsal sides of the penis,
wherein the vibrations are removed by moving the vibratory elements in directions perpendicular to the ventral and dorsal sides of the penis out of contact with the ventral and dorsal sides of the penis, and
wherein the vibratory elements are moved into and out of contact with the penis by the person whose penis is being stimulated.

36. A method of retarding chronic disease-related erectile dysfunction and degeneration and negative changes in penile nervous and erectile tissue, comprising:
simultaneously stimulating the nerves of the ventral and dorsal sides of the penis by simultaneously applying mechanical vibrations to the ventral and dorsal sides of the penis,
wherein the vibrations are applied by moving vibratory elements in directions perpendicular to the ventral and dorsal sides of the penis into contact with the ventral and dorsal sides of the penis,
wherein the vibrations are removed by moving the vibratory elements in directions perpendicular to the ventral and dorsal sides of the penis out of contact with the ventral and dorsal sides of the penis, and
wherein the vibratory elements are moved into and out of contact with the penis by the person whose penis is being stimulated.

37. The method according to claim 36, wherein the diseases to which the erectile dysfunction and degeneration and negative changes in penile nervous and erectile tissue are related is one or more of the group consisting of diabetes mellitus, chronic hypertension, peripheral vascular disease, high cholesterol, and atherosclerosis.
